(19) United States
(12) Patent Application Publication
(10) Pub. No.: US 2015/0031981 A1
(43) Pub. Date: Jan. 29, 2015

(54) HIGH-FREQUENCY ANTENNA UNIT AND A MAGNETIC RESONANCE APPARATUS WITH THE HIGH-FREQUENCY ANTENNA UNIT

(71) Applicant: Siemens Aktiengesellschaft, Munich (DE)

(72) Inventors: Yvonne CANDIDUS, Tuchenbach (DE); Ralf LADEBECK, Erlangen (DE)

(21) Appl. No.: 14/320,869

(22) Filed: Jul. 1, 2014

(30) Foreign Application Priority Data
Jul. 23, 2013 (DE) 102013214375.3

(51) Int. Cl. G01R 33/48; A61B 5/00; G01R 33/34

(52) U.S. Cl. CPC G01R 33/481; G01R 33/34; A61B 5/0035
USPC 600/411

(57) ABSTRACT

A high-frequency antenna unit includes a high-frequency antenna element and a stabilization layer, arranged at least partially around the one high-frequency antenna element. In at least one embodiment, the high-frequency antenna unit includes a layer which at least partially includes an imaging material.

### PRIORITY STATEMENT

The present application hereby claims priority under 35 U.S.C. §119 to German patent application number DE 102013214375.3 filed Jul. 23, 2013, the entire contents of which are hereby incorporated herein by reference.

### FIELD

At least one embodiment of the present invention generally relates to a high-frequency antenna unit having at least one high-frequency antenna element and a stabilization layer, arranged at least partially around the at least one high-frequency antenna element.

### BACKGROUND

Local high-frequency antenna units are frequently employed on a patient to detect high-frequency signals and/or magnetic resonance signals for magnetic resonance examinations in combination with a positron emission tomography examination (PET examination). However, the problem with this is that these local high-frequency antenna units, in particular flexible high-frequency antenna units, can be arranged at different positions on the patient and therefore also have different bending radii.

However, when combining magnetic resonance examinations with PET examinations as precise as possible a knowledge of a position and/or of an arrangement and/or of a bending radius of the local high-frequency antenna units is necessary in order to determine precisely a signal attenuation which photons in a PET examination experience when passing through matter, in particular the local high-frequency antenna units. If no account is taken of attenuation corrections, this can lead to PET events being missing from the PET data and/or to image artifacts in the reconstructed image data.

Until now flexible high-frequency antenna units have been constructed such that photons experience as little attenuation as possible when passing through matter. The low attenuation means that until now the local high-frequency antenna units have not been taken into account in an attenuation correction.

### SUMMARY

At least one embodiment of the present invention is directed to a high-frequency antenna unit which can be taken into account in an attenuation correction of a PET measurement. Advantageous embodiments are described in the subclaims.

At least one embodiment of the invention is based on a high-frequency antenna unit having at least one high-frequency antenna element and a stabilization layer, arranged at least partially around the at least one high-frequency antenna element.

In at least one embodiment, it is proposed that the high-frequency antenna unit should have a layer which at least partially includes an imaging material. This means that the high-frequency antenna unit, in particular a local high-frequency antenna unit for detecting high-frequency signals and/or magnetic resonance signals, can advantageously be detected and located during a magnetic resonance measurement and can be taken into account during a subsequent determination of an attenuation correction for correcting detected positron emission tomography data (PET data).

Furthermore, at least one embodiment of the invention is based on a combined imaging system having a magnetic resonance apparatus, a positron emission tomography apparatus and a high-frequency antenna unit, the high-frequency antenna unit comprising at least one high-frequency antenna element, a stabilization layer which is arranged at least partially around the at least one high-frequency antenna element, and a layer which at least partially contains an imaging material.

### BRIEF DESCRIPTION OF THE DRAWINGS

Further advantages, features and details of the invention will emerge from the example embodiments described hereinbelow as well as with reference to the drawings,

in which:

FIG. 1 shows a schematic representation of a combined imaging system having a high-frequency antenna unit,

FIG. 2 shows a schematic representation of the high-frequency antenna unit,

FIG. 3 shows a section through a first example embodiment of the high-frequency antenna unit,

FIG. 4 shows a section through a second example embodiment of the high-frequency antenna unit,

FIG. 5 shows a section through a third example embodiment of the high-frequency antenna unit and

FIG. 6 shows a section through a fourth example embodiment of the high-frequency antenna unit.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In at least one embodiment, it is proposed that the high-frequency antenna unit should have a layer which at least partially contains an imaging material. This means that the high-frequency antenna unit, in particular a local high-frequency antenna unit for detecting high-frequency signals and/or magnetic resonance signals, can advantageously be detected and located during a magnetic resonance measurement and can be taken into account during a subsequent determination of an attenuation correction for correcting detected positron emission tomography data (PET data).

In at least one embodiment, a three-dimensional location and/or orientation and/or extension of the high-frequency antenna unit can be detected in this way by way of a magnetic resonance measurement, regardless of a body region to be examined and/or the size of a patient. Preferably the high-frequency antenna unit is detected by way of a magnetic resonance measurement with a small echo time of approx. 1 ms. Advantageously the imaging material has a concentration within the layer such that a signal strength and/or signal intensity is sufficient to make the position of the high-frequency antenna unit visible in the image data of the magnetic resonance measurement, but does not affect the detection of medical image data.

In this connection a stabilization layer of a high-frequency antenna unit should in particular be understood as a layer which firstly protects the at least one high-frequency antenna element against damage and/or impairment and secondly gives the high-frequency antenna unit stability and simultaneously deformability, so that it is easy for medical staff to arrange. Preferably the stabilization layer is flexible, in particular deformable, in design, so that individual, interconnected subregions are designed to be deformable and/or movable in respect of one another as regards orientation and/or location. Furthermore, an imaging material should in particular be understood as a material which during a magnetic resonance measurement emits a high-frequency signal and/or a magnetic resonance signal and thus can be detected by way of the magnetic resonance measurement.

Furthermore, it is proposed that the high-frequency antenna unit should have at least one first antenna region and one second antenna region which are interconnected, the first antenna region being designed to be movable in respect of the second antenna region. In this way a particularly advantageous visibility of the high-frequency antenna unit can be achieved in the magnetic resonance data for in particular an embodiment of the high-frequency antenna unit with antenna regions that can be flexibly aligned to one another and/or arranged on the patient, since in particular in the case of such a high-frequency antenna unit it is particularly difficult to detect a location and/or orientation and/or positioning. Preferably the two antenna regions are here connected together.

A particularly space-saving and compact design of a high-frequency antenna unit can readily be achieved if the stabilization layer at least partially comprises the layer containing the imaging material. Preferably the stabilization layer is formed at least partially by the imaging material. The imaging material can here comprise a foam rubber and/or a neoprene and/or other materials which may seem expedient to the person skilled in the art, foam rubber and/or neoprene for example generating a magnetic resonance signal during a magnetic resonance measurement, which signal essentially has the same signal strength as a magnetic resonance signal of water.

Alternatively or additionally the stabilization layer can also comprise an adhesive layer which connects the at least one high-frequency antenna element to the stabilization layer, the adhesive layer at least partially comprising the layer containing the imaging material, as a result of which likewise a particularly space-saving and compact design of a high-frequency antenna unit can readily be achieved. Preferably the adhesive layer is formed at least partially by the imaging material. The adhesive layer can here comprise an adhesive film made of the imaging material.

Furthermore, in an alternative embodiment of the high-frequency antenna unit it is also conceivable for the high-frequency antenna unit to have an outer surface layer which outwardly shields the high-frequency antenna unit, the outer surface layer at least partially comprising the layer containing the imaging material. Here too a particularly space-saving and compact design of a high-frequency antenna unit can be readily achieved. Preferably the outer surface layer is here formed at least partially by the imaging material.

Moreover, depending on the configuration of the outer surface layer an advantageous cleanability of the high-frequency antenna unit can also be achieved, in particular if the outer surface layer is formed at least partially by a polyurethane textile, for example Plastibert 770S or Ploquet 1241, and/or a flexible paint layer, for example Baytec IMC. This advantageously also makes it easier to disinfect the high-frequency antenna unit.

A magnetic resonance signal which essentially has the same signal strength as a magnetic resonance signal of water can particularly advantageously be generated during a magnetic resonance measurement, for example by way of the PU textile and/or the flexible paint layer. Moreover, it is also conceivable for the outer surface layer to comprise Velcro tapes and/or fabric hook-and-loop tapes which are preferably formed from the imaging material.

A particularly advantageous detection of the layer containing the imaging material can be achieved if the high-frequency antenna unit has a receiving region for receiving a subregion of a patient, the outer surface layer being arranged on a side facing the receiving region. In this way an interfolding of interference signals can be prevented in the case of an upcoming magnetic resonance examination, since the side and/or surface layer facing the receiving region directly abuts the patient and therefore is arranged in particular within a field of view (FOV).

Further, the high-frequency antenna unit has an antenna support element, the antenna support element at least partially comprising the layer containing the imaging material, which means that a particularly space-saving and compact design of a high-frequency antenna unit can likewise readily be achieved. The antenna support element can here for example comprise a film which is arranged around the high-frequency antenna element or is glued to the high-frequency antenna element, the film comprising the imaging material. Moreover, other embodiments of the antenna support element are conceivable at any time.

In another embodiment of the invention it is proposed that the layer containing the imaging material should have a maximum layer thickness of 1.0 cm, in particular if the layer containing the imaging material is comprised at least partially of the stabilization layer. In this way a minimum layer thickness can be achieved, with sufficient visibility of the high-frequency antenna unit. Furthermore, in this way a layer thickness of an original function layer, for example the stabilization layer, can advantageously be retained and thus a particularly compact high-frequency antenna unit can be provided.

Particularly advantageously, the layer containing the imaging material here has a maximum layer thickness of 1 mm, which may in particular be advantageous if the layer is designed at least partially as an adhesive layer and/or as an outer surface layer, etc. In this way a layer thickness of an original function layer, for example the outer surface layer and/or the adhesive layer, can advantageously be retained and therefore a particularly compact high-frequency antenna unit can be provided.

Furthermore, it is proposed that the layer containing the imaging material covers at least 50% of a detection area of the high-frequency antenna unit. Particularly advantageously, however, the layer containing the imaging material covers at least 80% of the detection area of the high-frequency detection unit and particularly preferably virtually the entire detection area of the high-frequency antenna unit. Particularly good visibility of an extension of the high-frequency antenna unit can here be achieved in the magnetic resonance data and therefore account can also be taken of the extension of the high-frequency antenna unit during an attenuation value correction of the positron emission tomography data. Moreover, in this way any misinterpretation of the magnetic resonance data generated by the imaging material within the image data can be prevented.

Thus for example in the case of individual circular arrangements of the imaging material this could result in an interpretation of local tumor structures in the magnetic resonance image data. In this connection a detection area should in particular be understood as an area of the high-frequency antenna unit which at least encloses a high-frequency antenna element for the detection of high-frequency signals and/or magnetic resonance signals.

A particularly advantageous detection of the high-frequency antenna unit by way of a magnetic resonance measurement can be achieved if the layer containing the imaging material has a proton density, the proton density of the imaging material being smaller than a proton density of a subregion to be examined of a patient. In this way the high-frequency antenna unit, in particular an extension and/or a position of the high-frequency antenna unit, can be determined in the detected image data without thereby impairing a medical evaluation of the magnetic resonance data.

Particularly advantageously here, the imaging material is formed at least partially by a plastic, such as for example a foam rubber and/or a neoprene and/or a polyurethane tissue and/or a paint and/or Velcro elements and/or fabric hook-and-loop tapes and/or an adhesive material and/or a polysiloxane. For example, with imaging materials which are formed at least partially by a foam rubber and/or a neoprene and/or a polyurethane textile and/or a flexible paint, a magnetic resonance signal can be generated during a magnetic resonance measurement, the magnetic resonance signal having a similar signal strength to a signal strength of a magnetic resonance signal which is emitted by water.

Furthermore, at least one embodiment of the invention is based on a combined imaging system having a magnetic resonance apparatus, a positron emission tomography apparatus and a high-frequency antenna unit, the high-frequency antenna unit comprising at least one high-frequency antenna element, a stabilization layer which is arranged at least partially around the at least one high-frequency antenna element, and a layer which at least partially contains an imaging material.

In at least one embodiment, the high-frequency antenna unit, in particular a local high-frequency antenna unit for detecting high-frequency signals and/or magnetic resonance signals, can advantageously be detected and located during a magnetic resonance measurement and can be taken into account during a subsequent determination of an attenuation correction for correcting detected positron emission tomography data (PET data).

In particular, a three-dimensional location and/or orientation and/or extension of the high-frequency antenna unit can be detected in this way by way of a magnetic resonance measurement, regardless of the body region to be examined and/or size of a patient. Preferably the high-frequency antenna unit is detected by way of a magnetic resonance measurement with a small echo time of approx. 1 ms.

Additionally it is proposed that the combined imaging system has an evaluation unit which is designed to determine a position and/or location of the high-frequency antenna unit during an evaluation of magnetic resonance data and to take account of the determined position and/or location of the high-frequency antenna unit during a medical image reconstruction. In this way a spatial extension and/or a location of the high-frequency antenna unit during the image reconstruction of magnetic resonance data and particularly advantageously during the image reconstruction of positron emission tomography image data can be taken into account. In particular here a precise determination of attenuation values for an attenuation value correction of the positron emission tomography image data can be achieved, taking account of the extension and/or location of the high-frequency antenna unit.

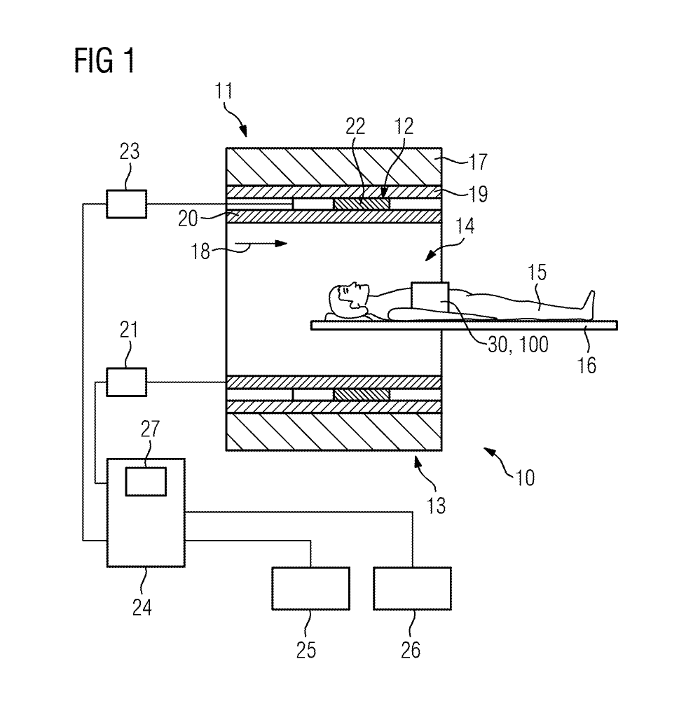

FIG. 1 shows a medical imaging system 10. The medical imaging system 10 is formed by a combined imaging system 10 which comprises a magnetic resonance apparatus 11 and a positron emission tomography apparatus 12 (PET apparatus 12).

The magnetic resonance apparatus 11 comprises a magnet unit 13 and a patient receiving region 14 surrounded by the magnet unit 13 to receive a patient 15, the patient receiving region 14 being surrounded cylindrically in a circumferential direction by the magnet unit 13. The patient 15 can be introduced into the patient receiving region 14 by way of a patient support apparatus 16 of the magnetic resonance apparatus 11. For this purpose the patient support apparatus 16 is arranged so as to be movable within the patient receiving region 16.

The magnet unit 13 comprises a main magnet 17 which during operation of the magnetic resonance apparatus 11 is designed to generate a strong and in particular constant main magnetic field 18. The magnet unit 13 additionally has a gradient coil unit 19 for generating magnetic field gradients which is used for spatial encoding during an imaging session. Moreover, the magnet unit 13 also has a first high-frequency antenna unit 20 which is formed by a high-frequency antenna transmit unit and which serves to stimulate a polarization which arises in the main magnetic field 18 generated by the main magnet 17. The first high-frequency antenna unit 20 is permanently integrated inside the magnet unit.

For the purpose of controlling the main magnet of the gradient coil unit 19 and of controlling the high-frequency antenna unit 20, the medical imaging system 10, in particular the magnetic resonance apparatus 11, has a control unit 21 formed by a computing unit. The control unit 21 is used for central control of the magnetic resonance apparatus 11, such as performing a predetermined imaging gradient echo sequence for example. To this end the control unit 21 comprises a gradient control unit (not shown in greater detail) and a high-frequency antenna control unit (not shown in greater detail). Moreover, the control unit 21 comprises an evaluation unit for evaluating magnetic resonance image data.

The magnetic resonance apparatus 11 shown can obviously comprise further components that magnetic resonance apparatuses typically include. Moreover, the general mode of operation of a magnetic resonance apparatus 11 is known to the person skilled in the art, so a detailed description of the general components will be dispensed with.

The PET apparatus 12 comprises several positron emission tomography detector modules 22 (PET detector modules 22) which are arranged in the form of a ring and surround the patient receiving region 14 in the circumferential direction. The PET detector modules 22 each have several positron emission tomography detector elements (PET detector elements, not shown in greater detail) which are arranged to form a PET detector array which comprises a scintillation detector array containing scintillation crystals, for example LSO crystals. Furthermore, the PET detector modules 22 each comprise a photodiode array, for example avalanche photodiode array or APD photodiode array, which are arranged downstream of the scintillation detector array inside the PET detector modules 22.

By way of the PET detector modules 22 pairs of photons resulting from the annihilation of a positron with an electron are detected. Trajectories of the two photons encompass an angle of 180°. Moreover, both the photons each have an energy of 511 keV. The positron is here emitted by a radiopharmaceutical, the radiopharmaceutical being administered to the patient 15 by way of an injection. When penetrating matter the photons produced by the annihilation may be absorbed, the probability of absorption depending on the path length through the matter and the corresponding absorption coefficient of the matter. Accordingly, when evaluating the PET signals it is necessary to correct these signals in respect of the attenuation by components situated in the radiation path.

Moreover, the PET detector modules 22 each have detector electronics which comprise an electrical amplifier circuit and other electronic components not shown in greater detail. For the purpose of controlling the detector electronics and the PET detector modules 22, the combined medical imaging system 10, in particular the PET apparatus 12, has another control unit 23 formed by a computing unit. The control unit 23 controls the PET apparatus 12 centrally. Moreover, the control unit 23 comprises an evaluation unit for evaluating PET data. The PET apparatus 12 shown can obviously comprise further components that PET apparatuses 12 typically include. Moreover, the general mode of operation of a PET apparatus 12 is known to the person skilled in the art, so a detailed description of the general components will be dispensed with.

The combined medical imaging system 10 moreover has a central system control unit 24 which for example coordinates detection and/or evaluation of magnetic resonance image data and PET image data with one another. Control information such as imaging parameters, for example, as well as reconstructed image data can be displayed on a display unit 25, for example on at least one monitor, of the combined medical imaging system 10 for viewing by an operator. Moreover, the combined medical imaging system 10 has an input unit 26 by way of which information and/or parameters can be entered by an operator during a measurement procedure.

In the present example embodiment, the magnetic resonance apparatus 11 comprises another high-frequency antenna unit 30 which is formed by a local high-frequency antenna receive unit and which is designed to receive magnetic resonance signals. The local high-frequency antenna unit 30 is applied around a body region to be examined of the patient 15 for a magnetic resonance examination by medical staff. In the present example embodiment the local high-frequency antenna unit 30 is formed by a body antenna unit. In principle an embodiment of the local high-frequency antenna unit 30 as a knee antenna unit and/or a back antenna unit, etc. is conceivable at any time.

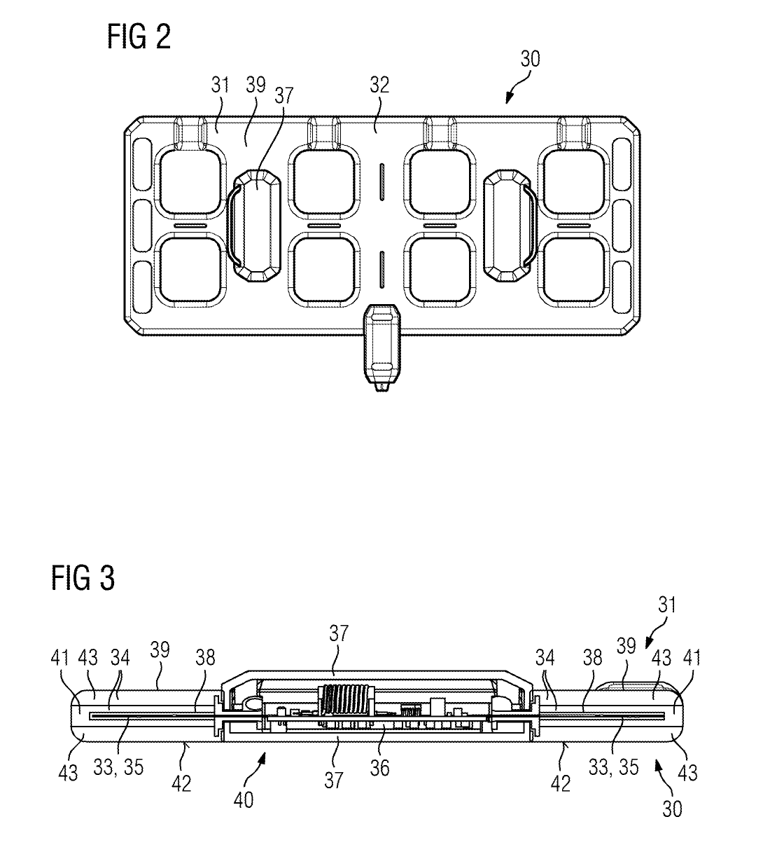

The high-frequency antenna unit 30 comprises several antenna regions 31, 32 which are connected to one another in a planar fashion (FIG. 2). The individual antenna regions 31, 32 are however designed to move relative to one another, so that when creating the local high-frequency antenna unit 30 they can be applied and/or arranged in an optimum position around the patient 15, in particular around the body region to be examined of the patient 15. The individual antenna regions 31, 32 of the local high-frequency antenna unit 30 each comprise a high-frequency antenna element 33 and a stabilization layer 34 which is arranged around the high-frequency antenna element 33, as shown in FIG. 3, which shows a section through the local high-frequency antenna unit 30.

The high-frequency antenna element 33 is here arranged on an antenna support element 35 of the high-frequency antenna unit 30 inside the high-frequency antenna unit 30. The antenna support element 35 is formed for example by a stabilization film which is applied to the high-frequency antenna element, and/or by further support elements of the high-frequency antenna element 33 which seem expedient to the person skilled in the art. Moreover, the stabilization layer 34 in the region of the high-frequency antenna element 33 has two subregions, the high-frequency antenna element 33 being arranged at least partially, in particular at edge regions of the high-frequency antenna element 33, between the two subregions of the stabilization layer 34. In a central region of the high-frequency antenna element 33 the high-frequency antenna unit 30 in each case has antenna electronics 36, the antenna electronics 36 together with the central region of the high-frequency antenna element 33 being surrounded by a fixed housing 37 of the high-frequency antenna unit 30.

Furthermore, the high-frequency antenna unit 30 has an adhesive layer 38 which is arranged between the high-frequency antenna element 33 and the individual subregions of the stabilization layer 34. By way of the adhesive layer 38, the high-frequency antenna element 33 is connected, in particular glued, to the stabilization layer 34. Furthermore, the high-frequency antenna unit 30 comprises an outer surface layer 39 which outwardly shields or protects the high-frequency antenna unit 30. The outer surface layer 39 is arranged at an outwardly facing surface of the stabilization layer 34.

The high-frequency antenna unit 30 also has a receiving region 40 for receiving a subregion to be examined of the patient 15. With a surface 39, facing this receiving region 40, of the high-frequency antenna unit 30 the high-frequency antenna unit 30 is applied to the patient 15 for the upcoming magnetic resonance examination.

The high-frequency antenna unit 30 further comprises a layer 41 which at least partially contains an imaging material. This means that it is possible to locate precisely where the high-frequency antenna unit 30 is during a magnetic resonance measurement, so that the high-frequency antenna unit 30 can be detected precisely for an attenuation correction of positron emission tomography signals (PET signals).

In order not to impair a medical imaging examination, the layer 41 containing the imaging material to this end has a proton density that is smaller than a proton density of the human body to be examined of the patient 15. This means that it is possible to avoid confusion between tumor tissue and the contours of the high-frequency antenna unit 30 which are visible in the magnetic resonance image data and therefore also to prevent impairment to and/or interference with a diagnosis.

Furthermore, the layer 41 containing the imaging material covers at least 50% of a detection area 42 of the high-frequency antenna unit 30. Particularly advantageously the layer 41 containing the imaging material covers at least 80% of the detection area 42 and particularly preferably the layer 41 containing the imaging material essentially completely covers the detection area 42.

In the present example embodiment, the stabilization layer 34 comprises the layer 41 containing the imaging material, so that the design of the high-frequency antenna unit 30 is particularly compact. Besides a function of stabilizing the high-frequency antenna unit 30 the stabilization layer 34 therefore has a further function of making the high-frequency antenna unit 30 visible in magnetic resonance image data. The layer 41 containing the imaging material is here formed at least partially by a plastic. In the present example embodiment the layer 41 containing the imaging material is formed by a foam rubber layer.

The stabilization layer 34 moreover has another layer 43, which is formed by a polyethylene foam layer (PE foam layer), so that the stabilization layer 34 is essentially composed of the foam rubber layer and the PE foam layer. The layer 41 containing the imaging material or the foam rubber layer is here arranged between the adhesive layer 28 and the PE foam layer of the stabilization layer 34. The stabilization layer 34 could also be entirely formed by the layer 41 containing the imaging material, so that the stabilization layer 34 comprises only the foam rubber layer.

Alternatively or additionally a neoprene layer would also be conceivable at any time to form the stabilization layer 34 or the layer 41 containing the imaging material. Moreover, other materials seeming expedient to the person skilled in the art would also be conceivable at any time to form the stabilization layer 34 or the layer 41 containing the imaging material.

By way of the imaging materials, which comprise a foam rubber and/or a neoprene and/or another material seeming expedient to the person skilled in the art, a magnetic resonance signal can be generated during a magnetic resonance measurement, the magnetic resonance signal having a similar signal strength to a signal strength of a magnetic resonance signal which is emitted by water.

Because the stabilization layer 34 is embodied with a maximum layer thickness of 1.0 cm, the layer 41 containing the imaging material also has a maximum layer thickness of 1.0 cm. In the present example embodiment, however, the foam rubber layer has a maximum layer thickness of approx. 0.5 cm.

Because the high-frequency antenna unit 30 is embodied with the layer 41 containing the imaging material, visibility of the high-frequency antenna unit 30 in magnetic resonance images of magnetic resonance measurements is achieved. In the present example embodiment visibility in magnetic resonance images of the stabilization layer 34 is achieved by way of the foam rubber layer. Preferably a contour of the high-frequency antenna unit 30 is detected by way of a magnetic resonance measurement with a small echo time of approx. 1 ms, as with magnetic resonance measurements with an f13d_ce sequence for example.

The central system control unit 24 of the combined medical imaging system 10 moreover has an evaluation unit 27 which on the basis of the magnetic resonance data detected determines precise positioning data and/or location data of a positioning and/or location of the high-frequency antenna unit 30. Based on this position data and/or location data of the high-frequency antenna unit 30 and information about a configuration and/or a material property of the high-frequency antenna unit 30, attenuation values are calculated by the evaluation unit 27 for an attenuation which photons experience when penetrating the high-frequency antenna unit 30 during PET data detection. These attenuation values are integrated by the evaluation unit 27 into an attenuation value map which is used for image reconstruction of the PET data of the PET measurement.

Furthermore, the magnetic resonance signals emitted and detected by the stabilization layer 34 are taken into account by the evaluation unit 27 during an evaluation, in particular an image reconstruction, of the magnetic resonance image data detected. In this way undesired interfolding during the image reconstruction can be advantageously prevented.

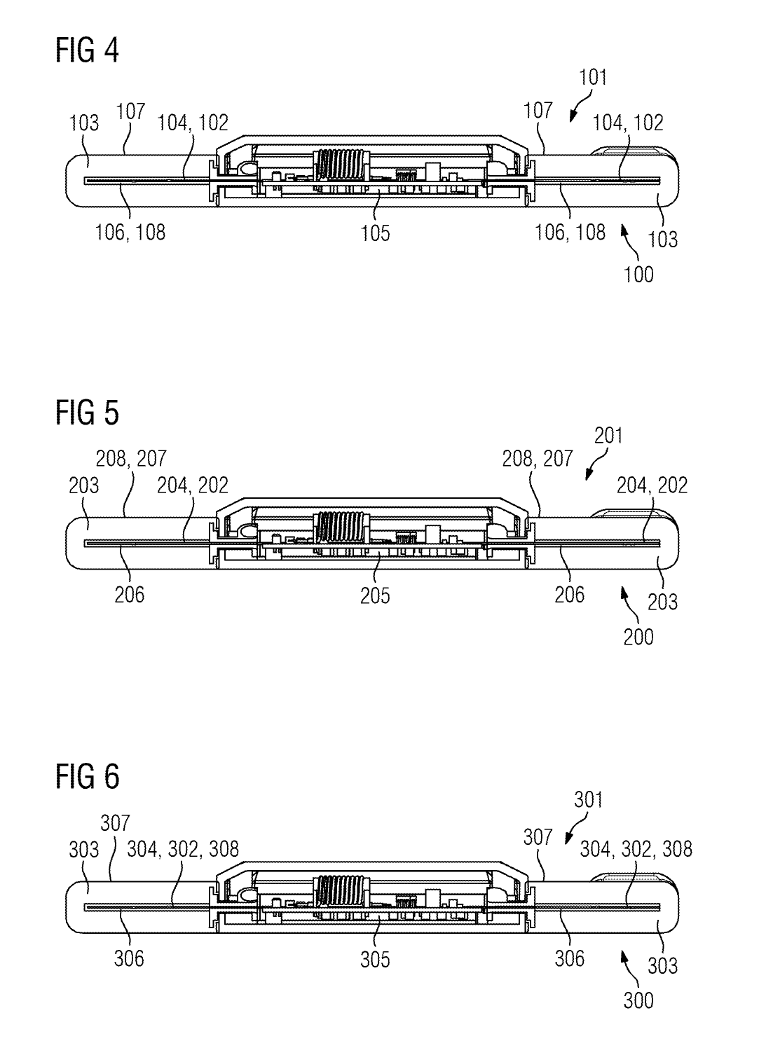

FIGS. 4 to 6 show alternative example embodiments of the high-frequency antenna unit 30. Components, features and functions remaining substantially the same are basically labeled with the same reference characters. The following description is essentially limited to the differences from the example embodiment shown in FIG. 3, with reference being made to the description of the example embodiment shown in FIG. 3 in respect of components, features and functions remaining the same.

FIG. 4 shows a section through a high-frequency antenna unit 100 designed as an alternative to FIG. 3. The high-frequency antenna unit 100 comprises, similarly to the example embodiment in FIGS. 2 and 3, several antenna regions 101, the individual antenna regions 101 of the high-frequency antenna unit 100 each comprising a high-frequency antenna element 102 and a stabilization layer 103 which is arranged around the high-frequency antenna element 102. The high-frequency antenna element 102 is here arranged on an antenna support element 104 of the high-frequency antenna unit inside the high-frequency antenna unit 100. Moreover, the stabilization layer 103 in the region of the high-frequency antenna element 102 has two subregions, the high-frequency antenna element 102 being arranged at least partially, in particular at edge regions of the high-frequency antenna element 102, between the two subregions of the stabilization layer 103. In a central region of the high-frequency antenna element 102 the high-frequency antenna unit 100 in each case has antenna electronics 105.

Furthermore, the high-frequency antenna unit 100 has an adhesive layer 106 which connects, in particular glues, the high-frequency antenna element 102 to the stabilization layer 103. The high-frequency antenna unit 100 further comprises an outer surface layer 107 which outwardly shields or protects the high-frequency antenna unit 100.

The high-frequency antenna unit 100 further comprises a layer 108 which at least partially contains an imaging material. In the present example embodiment the adhesive layer 106 comprises the layer 108 containing the imaging material. For example, the adhesive layer 106 or the layer 108 containing the imaging material is here formed by a contact adhesive containing imaging components. The layer 108 containing the imaging material is here designed to be particularly flexible, so that flexibility and/or deformability of the high-frequency antenna unit 100 is also retained.

Because the adhesive layer 106 within the high-frequency antenna unit 100 is designed to be thin, the layer 108 containing the imaging material is also designed to be thin. The adhesive layer 106 or the layer 108 containing the imaging material here has a maximum layer thickness of 1 mm.

FIG. 5 shows a section through a high-frequency antenna unit 200 designed as an alternative to FIGS. 3 and 4. The high-frequency antenna unit 200 comprises, similarly to the example embodiment in FIGS. 2 to 4, several antenna regions 201, the individual antenna regions 201 of the high-frequency antenna unit 200 each comprising a high-frequency antenna element 202 and a stabilization layer 203, which are arranged around the high-frequency antenna element 200. The high-frequency antenna element 202 is here arranged on an antenna support element 204 of the high-frequency antenna unit 200 inside the high-frequency antenna unit 200. Moreover, the stabilization layer 203 in the region of the high-frequency antenna element 202 has two subregions, the high-frequency antenna element 202 being arranged at least partially, in particular at edge regions of the high-frequency antenna element 202, between the two subregions of the stabilization layer 203. In a central region of the high-frequency antenna element 202 the high-frequency antenna unit 200 in each case has antenna electronics 205.

Furthermore, the high-frequency antenna unit 200 has an adhesive layer 206 which connects, in particular glues, the high-frequency antenna element 202 to the stabilization layer 203. The high-frequency antenna unit 200 further comprises an outer surface layer 207 which outwardly shields or protects the high-frequency antenna unit 200.

The high-frequency antenna unit 200 further comprises a layer 208 which at least partially contains an imaging material. In the present example embodiment the outer surface layer 207 comprises the layer 208 containing the imaging material. For example, the outer surface layer 207 or the layer 208 containing the imaging material is here formed by a polyurethane textile (PU textile), such as Plastibert 770S or Ploquet 1241. The layer 208 containing the imaging material is here designed to be particularly flexible, so that flexibility and/or deformability of the high-frequency antenna unit 200 is also retained.

Alternatively or additionally to the PU textile, the outer surface layer 207 or the layer 208 containing the imaging material can also have a flexible paint, for example Baytec IMC. These materials advantageously enable the high-frequency antenna unit 200 to be disinfected. Moreover, these materials also impart a high-quality appearance to the high-frequency antenna unit 200.

Alternatively or additionally to this, the outer surface layer 207 or the layer 208 containing the imaging material may also comprise Velcro tapes and/or fabric hook-and-loop tapes which likewise generate a weak magnetic resonance signal during a magnetic resonance measurement.

By way of the imaging materials, which comprise a PU textile and/or a flexible paint and/or another material seeming expedient to the person skilled in the art, a magnetic resonance signal can be generated during a magnetic resonance measurement, the magnetic resonance signal having a similar signal strength to a signal strength of a magnetic resonance signal which is emitted by water.

Because the outer surface layer 207 within the high-frequency antenna unit 200 is designed to be thin, the layer 208 containing the imaging material is also designed to be thin. The outer surface layer 207 or the layer 208 containing the imaging material here has a maximum layer thickness of 1 mm.

Preferably the outer surface layer 207 of the high-frequency antenna unit, which borders a receiving region for receiving the subregion to be examined of the patient 15, comprises the layer 208 containing the imaging material.

FIG. 6 shows a section through a high-frequency antenna unit 300 designed as an alternative to FIGS. 3 to 5. The high-frequency antenna unit 300 comprises, similarly to the example embodiment in FIGS. 2 and 3, several antenna regions 301, the individual antenna regions 301 of the high-frequency antenna unit 300 each comprising a high-frequency antenna element 302 and a stabilization layer 303 which is arranged around the high-frequency antenna element 302. The high-frequency antenna element 302 is here arranged on an antenna support element 304 of the high-frequency antenna unit 300 inside the high-frequency antenna unit 300.

Moreover, the stabilization layer 303 in the region of the high-frequency antenna element 302 has two subregions, the high-frequency antenna element 302 being arranged at least partially, in particular at edge regions of the high-frequency antenna element 302, between the two subregions of the stabilization layer 303. In a central region of the high-frequency antenna element 302 the high-frequency antenna unit 300 in each case has antenna electronics 305.

Furthermore, the high-frequency antenna unit 300 has an adhesive layer 306 which connects, in particular glues, the high-frequency antenna element 302 to the stabilization layer 303. The high-frequency antenna unit 300 further comprises an outer surface layer 307 which outwardly shields or protects the high-frequency antenna unit 300.

The high-frequency antenna unit 300 further comprises a layer 308 which at least partially contains an imaging material. In the present example embodiment the antenna support element 304 comprises the layer 308 containing the imaging material. For example, the antenna support element 304 or the layer 308 containing the imaging material here comprises a polysiloxane and/or the antenna support element 304 or the layer 308 containing the imaging material comprises an imaging paint layer, it being possible to coat the high-frequency antenna element 302 with the imaging paint layer. Moreover, the layer 308 containing the imaging material is here designed to be particularly flexible, so that flexibility and/or deformability of the high-frequency antenna unit 300 is also retained.

Because the antenna support element 304 within the high-frequency antenna unit 300 is designed to be thin, the layer 308 containing the imaging material is also designed to be thin. The antenna support element 304 or the layer 308 containing the imaging material here has a maximum layer thickness of 1 mm.

The high-frequency antenna units in FIGS. 4 to 6 are, in respect of an embodiment with several antenna regions, designed similarly to the description for FIGS. 2 and 3.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Although the invention has been illustrated and described in greater detail on the basis of the preferred example embodiments, the invention is not limited by the disclosed examples and other variations can be derived herefrom by the person skilled in the art without departing from the scope of protection of the invention.

What is claimed is:

1. A high-frequency antenna unit, comprising:
at least one high-frequency antenna element;
a stabilization layer, arranged at least partially around the at least one high-frequency antenna element; and
a layer, at least partially including an imaging material.

2. The high-frequency antenna unit of claim 1, further comprising:
at least one first antenna region and a second antenna region connected to one another, the first antenna region being embodied as movable with respect of the second antenna region.

3. The high-frequency antenna unit of claim 1, wherein the stabilization layer at least partially comprises the layer at least partially including the imaging material.

4. The high-frequency antenna unit of claim 1, further comprising:
an adhesive layer, connecting the at least one high-frequency antenna element to the stabilization layer, the adhesive layer at least partially comprising the layer at least partially including the imaging material.

5. The high-frequency antenna unit of claim 1, further comprising:
an outer surface layer, outwardly shielding the high-frequency antenna unit, the outer surface layer at least partially comprising the layer at least partially including the imaging material.

6. The high-frequency antenna unit of claim 5, further comprising:
a receiving region for receiving a partial region of a patient, the outer surface layer being arranged on a side facing the receiving region.

7. The high-frequency antenna unit of claim 1, further comprising:
an antenna support element, the antenna support element at least partially comprising the layer at least partially including the imaging material.

8. The high-frequency antenna unit of claim 1, wherein the layer at least partially including the imaging material includes a maximum layer thickness of 1.0 cm.

9. The high-frequency antenna unit of claim 1, wherein the layer at least partially including the imaging material includes a maximum layer thickness of 1 mm.

10. The high-frequency antenna unit of claim 1, wherein the layer covers at least 50% of a detection area of the high-frequency antenna unit.

11. The high-frequency antenna unit of claim 1, wherein the layer at least partially including the imaging material includes a proton density, the proton density of the imaging material being relatively smaller than a proton density of a subregion to be examined of a patient.

12. The high-frequency antenna unit of claim 1, wherein the imaging material is formed at least partially by a plastic.

13. A combined imaging system comprising:
a magnetic resonance apparatus;
a positron emission tomography apparatus; and
the high-frequency antenna unit of claim 1.

14. The combined imaging system of claim 13, further comprising:
evaluation unit, designed to determine at least one of a position and a location of the high-frequency antenna unit during an evaluation of magnetic resonance data and to take account of the determined at least one of position and location of the high-frequency antenna unit during a medical imaging reconstruction.

15. The high-frequency antenna unit of claim 2, wherein the stabilization layer at least partially comprises the layer at least partially including the imaging material.

16. The high-frequency antenna unit of claim 2, further comprising:
an adhesive layer, connecting the at least one high-frequency antenna element to the stabilization layer, the adhesive layer at least partially comprising the layer at least partially including the imaging material.

17. The high-frequency antenna unit of claim 2, further comprising:
an outer surface layer, outwardly shielding the high-frequency antenna unit, the outer surface layer at least partially comprising the layer at least partially including the imaging material.

18. The high-frequency antenna unit of claim 2, further comprising:
an antenna support element, the antenna support element at least partially comprising the layer at least partially including the imaging material.

19. A combined imaging system comprising:
a magnetic resonance apparatus;
a positron emission tomography apparatus; and
the high-frequency antenna unit of claim 2.

20. The combined imaging system of claim 19, further comprising:
evaluation unit, designed to determine at least one of a position and a location of the high-frequency antenna unit during an evaluation of magnetic resonance data and to take account of the determined at least one of position and location of the high-frequency antenna unit during a medical imaging reconstruction.
